(19) United States
(12) Patent Application Publication
(10) Pub. No.: US 2019/0365741 A1
(43) Pub. Date: Dec. 5, 2019

(54) COMBINATIONS OF THE CDK4/6 INHIBITOR LEE011 AND THE MEK1/2 INHIBITOR TRAMETINIB, OPTIONALLY FURTHER COMPRISING THE PI3K INHIBITOR BYL719 TO TREAT CANCER

(71) Applicant: Novartis AG, Basel (CH)

(72) Inventors: Giordano Caponigro, Cambridge, MA (US); Thomas Horn-Spirohn, Cambridge, MA (US); Joseph Lehar, Cambridge, MA (US)

(21) Appl. No.: 15/753,452

(22) PCT Filed: Aug. 24, 2016
(86) PCT No.: PCT/IB2016/055042

§ 371 Date: Feb. 19, 2018

Related U.S. Application Data
(60) Provisional application No. 62/211,026, filed on Aug. 28, 2015.

(51) Int. Cl. A61K 31/496; A61K 31/519; A61K 31/4439

(52) U.S. Cl. CPC A61K 31/496; A61K 31/4439; A61K 31/519

(57) ABSTRACT

The present disclosure relates to pharmaceutical combinations comprising a cyclin dependent kinase 4/6 (CDK4/6) inhibitor compound, (b) a mitogen activated protein kinase (MEK) inhibitor compound, and optionally (c) an alpha-isoform specific phosphatidylinositol 3-kinase (PI3K) inhibitor compound, for the treatment or prevention of cancer, as well as related pharmaceutical compositions, uses, and methods of treatment or prevention of cancer.

### TECHNICAL FIELD

The present disclosure relates to pharmaceutical combinations comprising a cyclin dependent kinase 4/6 (CDK4/6) inhibitor compound, (b) a mitogen activated protein kinase (MEK) inhibitor compound, and optionally (c) an alpha-isoform specific phosphatidylinositol 3-kinase (PI3K) inhibitor compound, for the treatment or prevention of cancer. The disclosure also provides related pharmaceutical compositions, uses, and methods of treatment or prevention of cancer.

### BACKGROUND

Tumor development is closely associated with genetic alteration and deregulation of cyclin dependent kinases (CDKs) and their regulators, suggesting that inhibitors of CDKs may be useful anti-cancer therapeutics. Indeed, early results suggest that transformed and normal cells differ in their requirement for, e.g., cyclin D/CDK4/6 and that it may be possible to develop novel antineoplastic agents devoid of the general host toxicity observed with conventional cytotoxic and cytostatic drugs.

The function of CDKs is to phosphorylate and thus activate or deactivate certain proteins, including, e.g., retinoblastoma proteins, lamins, histone H1, and components of the mitotic spindle. The catalytic step mediated by CDKs involves a phospho-transfer reaction from ATP to the macromolecular enzyme substrate. Several groups of compounds (reviewed in, e.g., Fischer, P. M. Curr. Opin. Drug Discovery Dev. 2001, 4, 623-634) have been found to possess anti-proliferative properties by virtue of CDK-specific ATP antagonism.

At a molecular level, mediation of CDK/cyclin complex activity requires a series of stimulatory and inhibitory phosphorylation, or dephosphorylation, events. CDK phosphorylation is performed by a group of CDK activating kinases (CAKs) and/or kinases such as wee1, Myt1 and Mik1. Dephosphorylation is performed by phosphatases such as Cdc25(a & c), PP2A, or KAP.

CDK/cyclin complex activity may be further regulated by two families of endogenous cellular proteinaceous inhibitors: the Kip/Cip family, or the INK family. The INK proteins specifically bind CDK4 and CDK6. p16^{ink4} (also known as MTS1) is a potential tumor suppressor gene that is mutated or deleted in a large number of primary cancers. The Kip/Cip family contains proteins such as p21^{Cip1,Waf1}, p27^{kip1} and p57^{kip2}, where p21 is induced by p53 and is able to inactivate the CDK2/cyclin(E/A) complex. Atypically low levels of p27 expression have been observed in breast, colorectal and prostate cancers. Conversely, over-expression of cyclin E in solid tumors has been shown to correlate with poor patient prognosis. Over-expression of cyclin D1 has been associated with esophageal, breast, squamous, and non-small cell lung carcinomas.

The pivotal roles of CDKs, and their associated proteins, in coordinating and driving the cell cycle in proliferating cells have been outlined above. Some of the biochemical pathways in which CDKs play a key role have also been described. The development of monotherapies for the treatment of proliferative disorders, such as cancers, using therapeutics targeted generically at CDKs, or at specific CDKs, is therefore potentially highly desirable. Thus, there is a continued need to find new therapeutic agents to treat human diseases.

Cell signaling through growth factor receptors and protein kinases is an important regulator of cell growth, proliferation and differentiation. In normal cell growth, growth factors (e.g., PDGF or EGF and others), through receptor activation, activate MAP kinase pathways. One of the most important and most well-understood MAP kinase pathways involved in normal and uncontrolled cell growth is the Ras/Raf kinase pathway. Active GTP-bound Ras results in the activation and indirect phosphorylation of Raf kinase. Raf then phosphorylates MEK1 and 2 on two serine residues (S218 and S222 for MEK1 and S222 and S226 for MEK2) (Ahn et al., Methods in Enzymology 2001, 332, 417-431). Activated MEK then phosphorylates its only known substrates, the MAP kinases ERK1 and ERK2. ERK phosphorylation by MEK occurs on Y204 and T202 for ERK1 and Y185 and T183 for ERK2 (Ahn et al., Methods in Enzymology 2001, 332, 417-431). Phosphorylated ERK dimerizes and then translocates to the nucleus where it accumulates (Khokhlatchev et al., Cell 1998, 93, 605-615). In the nucleus, ERK is involved in several important cellular functions, including but not limited to nuclear transport, signal transduction, DNA repair, nucleosome assembly and translocation, and mRNA processing and translation (Ahn et al., Molecular Cell 2000, 6, 1343-1354). Overall, treatment of cells with growth factors leads to the activation of ERK1 and 2 which results in proliferation and, in some cases, differentiation (Lewis et al., Adv. Cancer Res. 1998, 74, 49-139).

Receptor tyrosine kinases (RTKs) catalyze phosphorylation of certain tyrosine amino acid residues in various proteins, including themselves, which govern cell growth, proliferation and differentiation.

Downstream of the several RTKs lie several signaling pathways, among them is the Ras-Raf-MEK-ERK kinase pathway discussed above. It is currently understood that activation of Ras GTPase proteins in response to growth factors, hormones, cytokines, etc. stimulates phosphorylation and activation of Raf kinases. This signaling pathway, also known as the mitogen-activated protein kinase (MAPK) pathway or cytoplasmic cascade, mediates cellular responses to growth signals. The ultimate function of this signaling pathway is to link receptor activity at the cell membrane with modification of cytoplasmic or nuclear targets that govern cell proliferation, differentiation, and survival.

The constitutive activation of this pathway is sufficient to induce cellular transformation. Dysregulated activation of the MAP kinase pathway due to aberrant receptor tyrosine kinase activation, Ras mutations or Raf mutations has frequently been found in human cancers, and represents a major factor influencing abnormal growth control. In human malignances, Ras mutations are common, having been identified in about 30% of cancers. The Ras family of GTPase proteins (proteins which convert guanosine triphosphate to guanosine diphosphate) relay signals from activated growth factor receptors to downstream intracellular partners. Prominent among the targets recruited by active membrane-bound Ras are the Raf family of serine/threonine protein kinases. The Raf family is composed of three related kinases (A-, B- and C-Raf) that act as downstream effectors of Ras. Ras-medicated Raf activation, as discussed above, in turn triggers activation of MEK1 and MEK2 (MAP/ERK kinases 1 and 2) which in turn phosphorylate ERK1 and ERK2 (extracellular signal-regulated kinases 1 and 2) on the tyrosine-185 and threonine-183. Activated ERK1 and ERK2 translocate and accumulate in the nucleus, where they can phosphorylate a variety of substrates, including transcription factors that control cellular growth and survival. Given the importance of the Ras/Raf/MEK/ERK pathway in the development of human cancers, the kinase components of the signaling cascade are merging as potentially important targets for the modulation of disease progression in cancer and other proliferative diseases.

Mutations in various Ras GTPases and the B-Raf kinase have been identified that can lead to sustained and constitutive activation of the MAPK pathway, ultimately resulting in increased cell division and survival. As a consequence of this, these mutations have been strongly linked with the establishment, development, and progression of a wide range of human cancers.

MEK1 and MEK2 are members of a larger family of dual-specificity kinases (MEK1-7) that phosphorylate threonine and tyrosine residues of various MAP kinases. MEK1 and MEK2 are encoded by distinct genes, but they share high homology (80%) both within the C-terminal catalytic kinase domains and the most of the N-terminal regulatory region. Oncogenic forms of MEK1 and MEK2 have not been found in human cancers, but constitutive activation of MEK has been shown to result in cellular transformation. In addition to Raf, MEK can also be activated by other oncogenes as well. So far, the only known substrates of MEK1 and MEK2 are ERK1 and ERK2. This unusual substrate specificity in addition to the unique ability to phosphorylate both tyrosine and threonine residues places MEK1 and MEK2 at a critical point in the signal transduction cascade which allows it to integrate many extracellular signals into the MAPK pathway.

Accordingly, it has been recognized that an inhibitor of a protein of the MAPK kinase pathway (e.g., MEK) should be of value both as an anti-proliferative, pro-apoptotic and anti-invasive agent for use in the containment and/or treatment of proliferative or invasive disease. Moreover, it is also known that a compound having MEK inhibitory activity effectively induces inhibition of ERK1/2 activity and suppression of cell proliferation (The Journal of Biological Chemistry, vol. 276, No. 4 pp. 2686-2692, 2001), and the compound is expected to show effects on diseases caused by undesirable cell proliferation, such as tumor genesis and/or cancer.

Phosphatidylinositol 3-kinases (PI3Ks) comprise a family of lipid kinases that catalyze the transfer of phosphate to the D-3′ position of inositol lipids to produce phosphoinositol-3-phosphate (PIP), phosphoinositol-3,4-diphosphate (PIP2) and phosphoinositol-3,4,5-triphosphate (PIP₃) that, in turn, act as second messengers in signaling cascades by docking proteins containing pleckstrin-homology, FYVE, Phox and other phospholipid-binding domains into a variety of signaling complexes often at the plasma membrane ((Vanhaesebroeck et al., Annu. Rev. Biochem 70:535 (2001); Katso et al., Annu. Rev. Cell Dev. Biol. 17:615 (2001)). Of the two Class 1 PI3Ks, Class 1A PI3Ks are heterodimers composed of a catalytic p110 subunit (α, β, δ isoforms) constitutively associated with a regulatory subunit that can be p85α, p55α, p50α, p85β or p55γ. The Class 1B sub-class has one family member, a heterodimer composed of a catalytic p110γ subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor tyrosine kinases and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens et al., Cell 89:105 (1997)); Katso et al., Annu. Rev. Cell Dev. Biol. 17:615-675 (2001)). Consequently, the resultant phospholipid products of class I PI3K link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)).

In many cases, PIP₂ and PIP₃ recruit Akt, the product of the human homologue of the viral oncogene v-Akt, to the plasma membrane where it acts as a nodal point for many intracellular signaling pathways important for growth and survival (Fantl et al., Cell 69:413-423(1992); Bader et al., Nature Rev. Cancer 5:921 (2005); Vivanco and Sawyer, Nature Rev. Cancer 2:489 (2002)). Aberrant regulation of PI3K, which often increases survival through Akt activation, is one of the most prevalent events in human cancer and has been shown to occur at multiple levels. The tumor suppressor gene PTEN, which dephosphorylates phosphoinositides at the 3′ position of the inositol ring and in so doing antagonizes PI3K activity, is functionally deleted in a variety of tumors. In other tumors, the genes for the p110α isoform, PIK3CA, and for Akt are amplified and increased protein expression of their gene products has been demonstrated in several human cancers.

Furthermore, mutations and translocation of p85α that serve to up-regulate the p85-p110 complex have been described in human cancers. Finally, somatic missense mutations in PIK3CA that activate downstream signaling pathways have been described at significant frequencies in a wide diversity of human cancers (Kang at el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase and the upstream and downstream components of this signaling pathway is one of the most common deregulations associated with human cancers and proliferative diseases (Parsons et al., Nature 436:792 (2005); Hennessey at el., Nature Rev. Drug Disc. 4:988-1004 (2005)).

It has been found that the 2-carboxamide cycloamino urea derivatives of the formula (III) given below have advantageous pharmacological properties and inhibit, for example, PI3K (phosphatidylinositol 3-kinase). In particular, these compounds preferably show an improved selectivity for PI3K alpha with respect to beta and/or, delta and/or gamma subtypes. Hence, the compounds of formula (III) are suitable, for example, to be used in the treatment of diseases depending on PI3 kinases (in particular PI3K alpha, such as those showing overexpression or amplification of PI3K alpha or somatic mutation of PIK3CA), especially proliferative diseases such as tumor diseases and leukemias.

Further, these compounds preferably show improved metabolic stability and hence reduced clearance, leading to improved pharmacokinetic profiles.

In spite of numerous treatment options for cancer patients, there remains a need for effective and safe therapeutic agents and a need for their preferential use in combination therapy. In particular, there is a need for effective methods of treating cancers, especially those cancers that have been resistant and/or refractive to current therapies.

### SUMMARY

In a first aspect, provided herein is a pharmaceutical combination comprising:

(a) a first compound having the structure of formula (I):

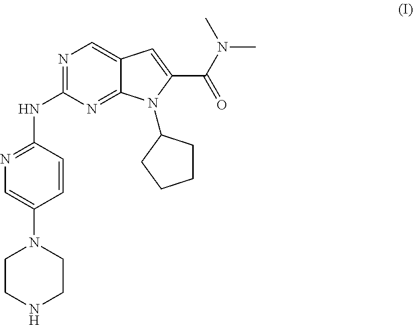 [SMILES: CN(C)C(=O)c1cc2cnc(Nc3ccc(N4CCNCC4)cn3)nc2n1C1CCCC1]

or a pharmaceutically acceptable salt or solvate thereof, and

(b) a second compound having the structure of formula (II):

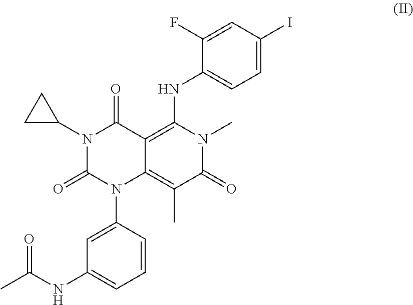 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt or solvate thereof.

In an embodiment, the compound having the structure of formula (I), or a pharmaceutically acceptable salt or solvate thereof, and the compound having the structure of formula (II), or a pharmaceutically acceptable salt or solvate thereof, are in the same formulation.

In an embodiment, the compound having the structure of formula (I), or a pharmaceutically acceptable salt or solvate thereof, and the compound having the structure of formula (II), or a pharmaceutically acceptable salt or solvate thereof, are in separate formulations.

In an embodiment, the combination of the first aspect is for simultaneous or sequential administration.

In an embodiment of the first aspect, the pharmaceutical combination further comprises a third compound having the structure of formula (III):

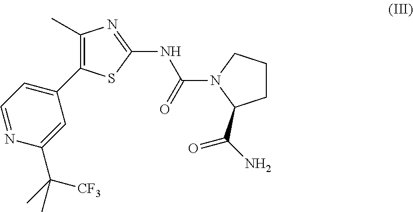 [SMILES: Cc1nc(NC(=O)N2CCC[C@H]2C(N)=O)sc1-c1ccnc(C(C)(C)C(F)(F)F)c1]

or a pharmaceutically acceptable salt or solvate thereof.

In an embodiment, the compound having the structure of formula (I), or a pharmaceutically acceptable salt or solvate thereof, the compound having the structure of formula (II), or a pharmaceutically acceptable salt or solvate thereof, and the compound having the structure of formula (III), or a pharmaceutically acceptable salt or solvate thereof, are in the same formulation.

In an embodiment, the compound having the structure of formula (I), or a pharmaceutically acceptable salt or solvate thereof, the compound having the structure of formula (II), or a pharmaceutically acceptable salt or solvate thereof, and the compound having the structure of formula (III), or a pharmaceutically acceptable salt or solvate thereof, are in 2 or more separate formulations.

In an embodiment, the compound having the structure of formula (I), or a pharmaceutically acceptable salt or solvate thereof, the compound having the structure of formula (II), or a pharmaceutically acceptable salt or solvate thereof, and the compound having the structure of formula (III), or a pharmaceutically acceptable salt or solvate thereof, are in 2 or 3 separate formulations.

In an embodiment, the pharmaceutical combination comprising the compound having the structure of formula (I), or a pharmaceutically acceptable salt or solvate thereof, the compound having the structure of formula (II), or a pharmaceutically acceptable salt or solvate thereof, and the compound having the structure of formula (III), or a pharmaceutically acceptable salt or solvate thereof, is for simultaneous or sequential administration.

In a particular embodiment of the pharmaceutical combinations described supra, the first compound is the succinate salt of the compound having the structure of formula (I).

In a second aspect, provided herein is a method for the treatment or prevention of cancer in a subject in need thereof, comprising administering to the subject a therapeutically effective amount of a pharmaceutical combination according to any one of the embodiments described supra.

In an embodiment, the cancer is selected from the group consisting of pancreatic cancer, breast cancer, mantle cell lymphoma, non-small cell lung cancer, melanoma, colorectal cancer, esophageal cancer, liposarcoma, multiple myeloma, T-cell leukemia, gastric cancer, renal cell carcinoma, glioblastoma, hepatocellular carcinoma, lung cancer, and rhabdoid tumor.

In a particular embodiment, the cancer is pancreatic cancer, breast cancer, or mantle cell lymphoma.

In a particular embodiment, the cancer is mantle cell lymphoma.

In a particular embodiment, the cancer is rhabdoid tumor.

In a particular embodiment, the cancer is colorectal cancer.

In certain particular embodiments of the second aspect, the cancer is characterized by a PIK3CA mutation and/or a PIK3CA overexpression.

In a third aspect, provided herein is a pharmaceutical combination as described supra for use in the treatment or prevention of cancer.

In a fourth aspect, provided herein is a pharmaceutical combination as described supra for use in the manufacture of a medicament for the treatment or prevention of cancer.

In certain embodiments of the third and fourth aspects, the cancer is selected from the group consisting of pancreatic cancer, breast cancer, mantle cell lymphoma, non-small cell lung cancer, melanoma, colorectal cancer, esophageal cancer, liposarcoma, multiple myeloma, T-cell leukemia, renal cell carcinoma, gastric cancer, glioblastoma, hepatocellular carcinoma, lung cancer, and rhabdoid tumor.

In a particular embodiment, the cancer is pancreatic cancer, breast cancer, or mantle cell lymphoma.

In a particular embodiment, the cancer is mantle cell lymphoma.

In a particular embodiment, the cancer is rhabdoid tumor.

In a particular embodiment, the cancer is colorectal cancer.

In certain particular embodiments of the third and fourth aspects, the cancer is characterized by a PIK3CA mutation and/or PIK3CA overexpression.

In a fifth aspect, provided herein is the use of a pharmaceutical combination as described supra for the manufacture of a medicament for the treatment or prevention of cancer.

In a sixth aspect, provided herein is the use of a pharmaceutical combination as described supra for the treatment or prevention of cancer.

In particular embodiments of the fifth and sixth aspects, the cancer is selected from the group consisting of pancreatic cancer, breast cancer, mantle cell lymphoma, non-small cell lung cancer, melanoma, colorectal cancer, esophageal cancer, liposarcoma, multiple myeloma, T-cell leukemia, renal cell carcinoma, gastric cancer, glioblastoma, hepatocellular carcinoma, lung cancer, and rhabdoid tumor.

In a particular embodiment, the cancer is pancreatic cancer, breast cancer, or mantle cell lymphoma.

In a particular embodiment, the cancer is mantle cell lymphoma.

In a particular embodiment, the cancer is rhabdoid tumor.

In a particular embodiment, the cancer is colorectal cancer.

In certain particular embodiments of the fifth and sixth aspects, the cancer is characterized by a PIK3CA mutation and/or PIK3CA overexpression.

In a seventh aspect, provided herein is a pharmaceutical composition comprising:
(a) a first compound having the structure of formula (I):

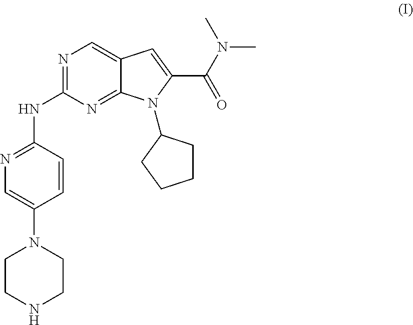 [SMILES: CN(C)C(=O)c1cc2cnc(Nc3ccc(N4CCNCC4)cn3)nc2n1C1CCCC1]

or a pharmaceutically acceptable salt or solvate thereof, and
(b) a second compound having the structure of formula (II):

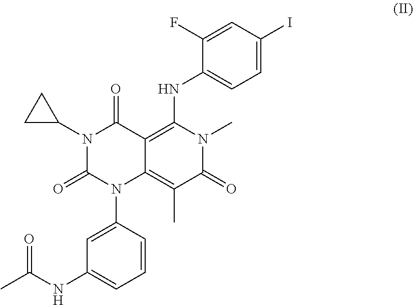 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt or solvate thereof.

In an embodiment of the seventh aspect, the pharmaceutical composition further comprises a third compound having the structure of formula (III):

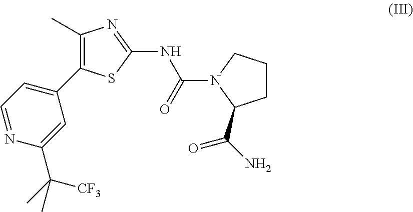 [SMILES: Cc1nc(NC(=O)N2CCC[C@H]2C(N)=O)sc1-c1ccnc(C(C)(C)C(F)(F)F)c1]

or a pharmaceutically acceptable salt or solvate thereof.

In an embodiment, the pharmaceutical composition comprises one or more excipients.

### BRIEF DESCRIPTION OF THE DRAWINGS

FIG. 1 shows dose-response curves for LEE011, trametinib, BYL719, and combinations thereof over 15 colorectal cancer cell lines. The x-axis indicates the log10 of the treatment dilution; the y-axis indicates the cell count after treatment relative to DMSO. The strong dashed line indicates the number of cells before the start of the treatment (‘baseline’).

FIG. 2 shows maximum Caspase 3/7 induction for LEE011, trametinib, BYL719, and combinations thereof in 15 colorectal cancer cell lines after 24 h, 48 h, and 72 h (different shades of grey). The x-axis indicates the treatment; the y-axis indicates the maximum Caspase 3/7 induction (% of cells) seen for each treatment.

FIG. 3 shows dose-response curves for LEE011, trametinib, and the combination of LEE011 and trametinib over 15 colorectal cancer cell lines. The x-axis indicates the log10 of the treatment dilution; the y-axis indicates the cell count after treatment relative to DMSO. The strong dashed line indicates the number of cells before the start of the treatment (‘baseline’).

FIG. 4 shows maximum Caspase 3/7 induction for LEE011, trametinib, and the combination of LEE011 and trametinib in 15 colorectal cancer cell lines after 24 h, 48 h, and 72 h (different shades of grey). The x-axis indicates the treatment; the y-axis indicates the maximum Caspase 3/7 induction (% of cells) seen for each treatment.

FIG. 5a shows representative images of cells after crystal violet staining following long-term colony formation assays for single agents and combination of LEE011 and trametinib. For all lines LEE011 was used at a dose of 3 μM; for DLD-1 and SW-480 trametinib was used at a dose of 33 nM, for HT-29 at a dose of 1.2 nM.

FIG. 5b shows quantification of crystal violet signal from FIG. 5a for triplicate measurements per condition (RFU=relative fluorescence unit), which indicates that for all cell lines the combination treatment has significantly less signal than each of single agent treatments (**p<0.01, ***p<0.001; one-tailed t-test).

### DETAILED DESCRIPTION

### Inhibitor Compounds

The CDK 4/6 inhibitor 7-Cyclopentyl-2-(5-piperazin-1-yl-pyridin-2-ylamino)-7H-pyrrolo[2,3-d]pyrimidine-6-carboxylic acid dimethylamide (also known as “LEE011” or “ribociclib”) is referred to herein as the compound having the structure of formula (I), or compound (I):

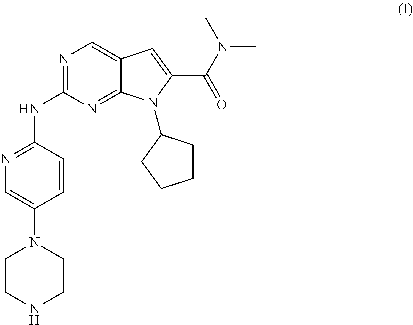 [SMILES: CN(C)C(=O)c1cc2cnc(Nc3ccc(N4CCNCC4)cn3)nc2n1C1CCCC1]

Compound (I), and pharmaceutically acceptable salts and solvates thereof are described in International Publication No. WO 2010/020675 (e.g., in Example 74), the entire contents of which is hereby incorporated by reference.

The MEK inhibitor N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide (also known as “trametinib”) is referred to herein as the compound having the structure of formula (II), or compound (II):

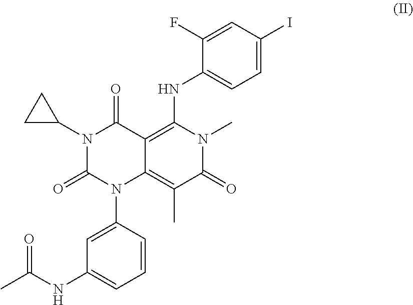 [SMILES: CC(=O)Nc1cccc(-n2c(=O)n(C3CC3)c(=O)c3c(Nc4ccc(I)cc4F)n(C)c(=O)c(C)c32)c1]

Compound (II), and pharmaceutically acceptable salts and solvates thereof are described in International Publication Number WO 2005/121142 (e.g., Example 4-1), the entire contents of which is hereby incorporated by reference.

The alpha-isoform specific PI3K inhibitor compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (also known as “BYL719” or “alpelisib”) is referred to herein as the compound having the structure of formula (III) , or compound (III):

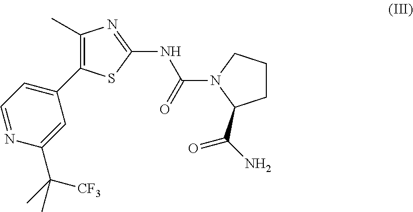 [SMILES: Cc1nc(NC(=O)N2CCC[C@H]2C(N)=O)sc1-c1ccnc(C(C)(C)C(F)(F)F)c1]

Compound (III), and pharmaceutically acceptable salts and solvates thereof are described in International Application No. WO 2010/029082 (e.g., Example 15). This publication is incorporated herein by reference in its entirety.

### Salts and Solvates

Salts of the inhibitor compounds described herein can be present alone or in a mixture with the free base form, and are preferably pharmaceutically acceptable salts. A “pharmaceutically acceptable salt”, as used herein, unless otherwise indicated, includes salts of acidic and basic groups which may be present in the compounds of the present invention. Such salts may be formed, for example, as acid addition salts, preferably with organic or inorganic acids, upon reaction with a basic nitrogen atom. Suitable inorganic acids are, for example, halogen acids, such as hydrochloric acid, sulfuric acid, or phosphoric acid. Suitable organic acids are, e.g., carboxylic acids or sulfonic acids, such as fumaric acid or methansulfonic acid. For isolation or purification purposes it is also possible to use pharmaceutically unacceptable salts, for example picrates or perchlorates.

In a preferred embodiment of the pharmaceutical combinations described herein, the compound having the structure of formula (I) is in the form of a succinate salt.

In a preferred embodiment of the pharmaceutical combinations described herein, the compound having the structure of formula (II) is in the form of a dimethyl sulfoxide solvate. In certain embodiments, the compound having the structure of formula (II) is in the form of a solvate selected from: hydrate, acetic acid, ethanol, nitromethane, chlorobenzene, 1-pentanol, isopropyl alcohol, ethylene glycol and 3-methyl-1-butanol. These solvates can be prepared by one of skill in the art from the description in International Publication Number WO 2005/121142 or United States Patent Publication No. US 2006/0014768.

In a preferred embodiment of the pharmaceutical combinations described herein, the compound having the structure of formula (III) is in the form of its free base.

For therapeutic use, only pharmaceutically acceptable salts, solvates or free compounds are employed (where applicable in the form of pharmaceutical preparations), and these are therefore preferred. In view of the close relationship between the compounds in their free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient. Salts contemplated herein are preferably pharmaceutically acceptable salts; suitable counter-ions forming pharmaceutically acceptable salts are known in the field.

### Pharmaceutical Combinations and Compositions

The combinations and compositions can be administered to a system comprising cells or tissues, as well as a human subject (e.g., a patient) or an animal subject.

The combination and composition of the present invention can be administered in various dosage forms and strength, in a pharmaceutically effective amount or a clinically effective amount.

The pharmaceutical compositions for separate administration of both combination components, or for the administration in a fixed combination, e.g., a single galenical composition comprising the combination, may be prepared in any manner known in the art and are those suitable for enteral, such as oral or rectal, and parenteral administration to mammals (warm-blooded animals), including humans.

The pharmaceutical compositions described herein may contain, from about 0.1% to about 99.9%, preferably from about 1% to about 60%, of the therapeutic agent(s). Suitable pharmaceutical compositions for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of various conventional mixing, comminution, direct compression, granulating, sugar-coating, dissolving, lyophilizing processes, or fabrication techniques readily apparent to those skilled in the art. It will be appreciated that the unit content of a combination partner contained in an individual dose of each dosage form need not in itself constitute an effective amount since the necessary effective amount may be reached by administration of a plurality of dosage units.

A unit dosage form containing the combination of agents or individual agents of the combination of agents may be in the form of micro-tablets enclosed inside a capsule, e.g. a gelatin capsule. For this, a gelatin capsule as is employed in pharmaceutical formulations can be used, such as the hard gelatin capsule known as CAPSUGEL, available from Pfizer.

The unit dosage forms of the present invention may optionally further comprise additional conventional carriers or excipients used for pharmaceuticals. Examples of such carriers include, but are not limited to, disintegrants, binders, lubricants, glidants, stabilizers, and fillers, diluents, colorants, flavours and preservatives. One of ordinary skill in the art may select one or more of the aforementioned carriers with respect to the particular desired properties of the dosage form by routine experimentation and without any undue burden. The amount of each carriers used may vary within ranges conventional in the art. The following references which are all hereby incorporated by reference disclose techniques and excipients used to formulate oral dosage forms. See The Handbook of Pharmaceutical Excipients, 4^{th} edition, Rowe et al., Eds., American Pharmaceuticals Association (2003); and Remington: the Science and Practice of Pharmacy, 20^{th} edition, Gennaro, Ed., Lippincott Williams & Wilkins (2003).

As used herein, the term “pharmaceutically acceptable excipient” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289- 1329). Except insofar as any conventional carrier is incompatible with the active ingredient, its use in the therapeutic or pharmaceutical compositions is contemplated.

These optional additional conventional carriers may be incorporated into the oral dosage form either by incorporating the one or more conventional carriers into the initial mixture before or during granulation or by combining the one or more conventional carriers with granules comprising the combination of agents or individual agents of the combination of agents in the oral dosage form. In the latter embodiment, the combined mixture may be further blended, e.g., through a V-blender, and subsequently compressed or molded into a tablet, for example a monolithic tablet, encapsulated by a capsule, or filled into a sachet.

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 5% by weight of composition.

Examples of pharmaceutically acceptable binders include, but are not limited to, starches; celluloses and derivatives thereof, for example, microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder may be present in an amount from about 0% to about 50%, e.g., 2-20% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 80% by weight of the composition.

The optimal dosage of each combination partner for treatment of cancer can be determined empirically for each individual using known methods and will depend upon a variety of factors, including, though not limited to, the degree of advancement of the disease; the age, body weight, general health, gender and diet of the individual; the time and route of administration; and other medications the individual is taking. Optimal dosages may be established using routine testing and procedures that are well known in the art.

The amount of each combination partner that may be combined with the carrier materials to produce a single dosage form will vary depending upon the individual treated and the particular mode of administration. In some embodiments the unit dosage forms containing the combination of agents as described herein will contain the amounts of each agent of the combination that are typically administered when the agents are administered alone.

The effective dosage of each of the combination partners employed in the combination of the invention may vary depending on the particular compound or pharmaceutical composition employed, the mode of administration, the condition being treated, and the severity of the condition being treated. Thus, the dosage regimen of the combinations described herein are selected in accordance with a variety of factors including the route of administration and the renal and hepatic function of the patient.

The effective dosage of each of the combination partners may require more frequent administration of one of the compound(s) as compared to the other compound(s) in the combination. Therefore, to permit appropriate dosing, packaged pharmaceutical products may contain one or more dosage forms that contain the combination of compounds, and one or more dosage forms that contain one of the combination of compounds, but not the other compound(s) of the combination.

Compound (I) (“LEE011” or “ribociclib”) (based on weight of the unsalted/unsolvated compound), in general, is administered in a dose in the range from 10 mg to 2000 mg per day in human. In one embodiment, LEE011 is administered 600 mg QD. In another embodiment, LEE011 is administered 300 mg QD. In another embodiment, LEE011 is administered in 900 mg QD.

Compound (II) (“trametinib”) (based on weight of unsalted/unsolvated amount) administered as part of the combination according to the present invention in human will be an amount selected from about 0.125 mg to about 10 mg per day; suitably, the amount will be selected from about 0.25 mg to about 9 mg per day; suitably, the amount will be selected from about 0.25 mg to about 8 mg; suitably, the amount will be selected from about 0.5 mg to about 8 mg per day; suitably, the amount will be selected from about 0.5 mg to about 7 mg per day; suitably, the amount will be selected from about 1 mg to about 5 mg per day; suitably, the amount will be about 2 mg per day.

Compound (III) (“BYL719” or “alpelisib”) may be orally administered at an effective daily dose of about 1 to 6.5 mg/kg in human adults or children. Compound (III) may be orally administered to a 70 kg body weight human adult at a daily dosage of about 70 mg to 455 mg, e.g., about 200 to 400 mg, or about 240 mg to 400 mg, or about 300 mg to 400 mg, or about 350 mg to 400 mg, in a single dose or in divided doses up to four times a day. Preferably, compound (III) is administered to a 70 kg body weight human adult at a daily dosage of about 350 mg to about 400 mg.

The optimum ratios, individual and combined dosages, and concentrations of the combination partners of the combination of the invention (i.e., compound (I), compound (II), and optionally compound (III)) that yield efficacy without toxicity are based on the kinetics of the therapeutic agents' availability to target sites, and are determined using methods known to those of skill in the art.

Frequency of dosage may vary depending on the compound used and the particular condition to be treated or prevented. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

In certain aspects, the pharmaceutical combinations described herein are useful for the treatment or prevention of cancer, or for the preparation of a medicament for the treatment or prevention of cancer. In a particular embodiment, the pharmaceutical combinations described herein are useful for the treatment of cancer, or for the preparation of a medicament for the treatment of cancer.

In certain aspects, a method for the treatment or prevention of cancer (e.g., for the treatment of cancer) is provided, comprising administering to a patient in need thereof a pharmaceutically effective amount of a pharmaceutical combination described herein. The nature of cancer is multifactorial. Under certain circumstances, drugs with different mechanisms of action may be combined. However, just considering any combination of therapeutic agents having different mode of action does not necessarily lead to combinations with advantageous effects.

In an embodiment, the cancer is selected from the group consisting of pancreatic cancer, breast cancer, mantle cell lymphoma, non-small cell lung cancer, melanoma, colorectal cancer, esophageal cancer, liposarcoma, multiple myeloma, T-cell leukemia, gastric cancer, renal cell carcinoma, glioblastoma, hepatocellular carcinoma, lung cancer, and rhabdoid tumor.

In a particular embodiment, the cancer is pancreatic cancer, breast cancer, or mantle cell lymphoma.

In a particular embodiment, the cancer is mantle cell lymphoma.

In a particular embodiment, the cancer is rhabdoid tumor.

In a particular embodiment, the cancer is colorectal cancer.

In certain particular embodiments of the second aspect, the cancer is characterized by a PIK3CA mutation and/or a PIK3CA overexpression.

In a third aspect, provided herein is a pharmaceutical combination as described supra for use in the treatment or prevention of cancer.

In a fourth aspect, provided herein is a pharmaceutical combination as described supra for use in the manufacture of a medicament for the treatment or prevention of cancer.

In certain embodiments of the third and fourth aspects, the cancer is selected from the group consisting of pancreatic cancer, breast cancer, mantle cell lymphoma, non-small cell lung cancer, melanoma, colorectal cancer, esophageal cancer, liposarcoma, multiple myeloma, T-cell leukemia, renal cell carcinoma, gastric cancer, glioblastoma, hepatocellular carcinoma, lung cancer, and rhabdoid tumor.

In a particular embodiment, the cancer is pancreatic cancer, breast cancer, or mantle cell lymphoma.

In a particular embodiment, the cancer is mantle cell lymphoma.

In a particular embodiment, the cancer is rhabdoid tumor.

In a particular embodiment, the cancer is colorectal cancer.

The administration of a pharmaceutical combination as described herein may result not only in a beneficial effect, e.g., a synergistic therapeutic effect, e.g., with regard to alleviating, delaying progression of or inhibiting the symptoms, but also in further surprising beneficial effects, e.g., fewer side-effects, a more durable response, an improved quality of life or a decreased morbidity, compared with a monotherapy applying only one of the pharmaceutically therapeutic agents used in the combination of the invention.

A further benefit is that lower doses of the therapeutic agents of a pharmaceutical combination as described herein can be used, for example, such that the dosages may not only often be smaller, but are also may be applied less frequently, or can be used in order to diminish the incidence of side-effects observed with one of the combination partners alone. This is in accordance with the desires and requirements of the patients to be treated.

It can be shown by established test models that a pharmaceutical combination as described herein results in the beneficial effects described herein before. The person skilled in the art is fully enabled to select a relevant test model to prove such beneficial effects. The pharmacological activity of a combination of the invention may, for example, be demonstrated in a clinical study or in an animal model.

Determining a synergistic interaction between one or more components, the optimum range for the effect and absolute dose ranges of each component for the effect may be definitively measured by administration of the components over different w/w ratio ranges and doses to patients in need of treatment. For humans, the complexity and cost of carrying out clinical studies on patients may render impractical the use of this form of testing as a primary model for synergy. However, the observation of synergy in certain experiments (see, e.g., examples 1 and 2) can be predictive of the effect in other species and animal models exist to further measure a synergistic effect. The results of such studies can also be used to predict effective dose ratio ranges and the absolute doses and plasma concentrations.

In an embodiment, the combinations and/or compositions provided herein display a synergistic effect.

In an embodiment, provided herein is a synergistic combination for administration to a human, said combination comprising the inhibitors described herein, where the dose range of each inhibitor corresponds to the synergistic ranges suggested in a suitable tumor model or clinical study.

When the combination partners, which are employed in the combination of the invention, are applied in the form as marketed as single drugs, their dosage and mode of administration can be in accordance with the information provided on the package insert of the respective marketed drug, if not mentioned herein otherwise.

### Definitions

Certain terms used herein are described below. Compounds are described using standard nomenclature. Unless defined otherwise, all technical and scientific terms used herein have the meaning that is commonly understood by one of skill in the art to which the present disclosure belongs.

The term “pharmaceutical composition” is defined herein to refer to a mixture or solution containing at least one therapeutic agent to be administered to a subject, e.g., a mammal or human, in order to prevent or treat a particular disease or condition affecting the mammal or human.

The term “pharmaceutically acceptable” is defined herein to refer to those compounds, materials, compositions and/or dosage forms, which are, within the scope of sound medical judgment, suitable for contact with the tissues a subject, e.g., a mammal or human, without excessive toxicity, irritation allergic response and other problem complications commensurate with a reasonable benefit/risk ratio.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present invention, the term “treat” also denotes to arrest, delay the onset (i.e., the period prior to clinical manifestation of a disease) and/or reduce the risk of developing or worsening a disease. The term “prevent”, “preventing” or “prevention” as used herein comprises the prevention of at least one symptom associated with or caused by the state, disease or disorder being prevented.

The term “pharmaceutically effective amount” or “clinically effective amount” of a combination of therapeutic agents is an amount sufficient to provide an observable improvement over the baseline clinically observable signs and symptoms of the disorder treated with the combination.

The term “combination,” “therapeutic combination,” or “pharmaceutical combination” as used herein refer to either a fixed combination in one dosage unit form, or non-fixed combination or a kit of parts for the combined administration where two or more therapeutic agents may be administered independently, at the same time, or separately within time intervals, especially where these time intervals allow that the combination partners to show a cooperative, e.g., synergistic, effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (e.g., capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the therapeutic agents are administered to the same patient as part of the same course of therapy. In any case, the treatment regimen will provide beneficial effects in treating the conditions or disorders described herein.

The term “synergistic effect” as used herein refers to action of two therapeutic agents such as, for example, the CDK inhibitor compound (I), and the MEK inhibitor compound (II), and optionally the PI3K inhibitor compound (III), producing an effect, for example, slowing the symptomatic progression of a proliferative disease, particularly cancer, or symptoms thereof, which is greater than the simple addition of the effects of each therapeutic agent administered alone. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

The term “subject” or “patient” as used herein includes animals, which are capable of suffering from or afflicted with a cancer or any disorder involving, directly or indirectly, a cancer. Examples of subjects include mammals, e.g., humans, dogs, cows, horses, pigs, sheep, goats, cats, mice, rabbits, rats and transgenic non-human animals. In the preferred embodiment, the subject is a human, e.g., a human suffering from, at risk of suffering from, or potentially capable of suffering from cancer.

The terms “fixed combination” and “fixed dose” and “single formulation” as used herein refer to single carrier or vehicle or dosage forms formulated to deliver an amount, which is jointly therapeutically effective for the treatment of cancer, of two or more therapeutic agents to a patient. The single vehicle is designed to deliver an amount of each of the agents, along with any pharmaceutically acceptable carriers or excipients. In some embodiments, the vehicle is a tablet, capsule, pill, or a patch. In other embodiments, the vehicle is a solution or a suspension.

The term “non-fixed combination,” “kit of parts,” and “separate formulations” means that the active ingredients, e.g., LEE011 and trametinib are both administered to a patient as separate entities either simultaneously, concurrently or sequentially with no specific time limits, wherein such administration provides therapeutically effective levels of the two compounds in the body of the warm-blooded animal in need thereof. The latter also applies to cocktail therapy, e.g., the administration of three or more active ingredients.

The term “unit dose” is used herein to mean simultaneous administration of two or three agents together, in one dosage form, to the patient being treated. In some embodiments, the unit dose is a single formulation. In certain embodiments, the unit dose includes one or more vehicles such that each vehicle includes an effective amount of at least one of the agents along with pharmaceutically acceptable carriers and excipients. In some embodiments, the unit dose is one or more tablets, capsules, pills, injections, infusions, patches, or the like, administered to the patient at the same time.

An “oral dosage form” includes a unit dosage form prescribed or intended for oral administration.

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “about” or “approximately” shall have the meaning of within 10%, more preferably within 5%, of a given value or range.

### EXAMPLES

### Materials and Methods

The compounds were dissolved in 100% DMSO (Sigma, Catalog number D2650) at concentrations of 20 mM and stored at −20° C. until use. Compounds were arrayed in drug master plates (Greiner, Catalog number 788876) and serially diluted 3-fold (7 steps) at 2000× concentration.

Colorectal cancer cell lines used for this study were obtained, cultured and processed from commercial vendors ATCC, CellBank Australia, DMSZ, ECACC, and HSRRB (Table 1). All cell line media were supplemented with 10% FBS (HyClone, Catalog number SH30071.03). Media for LIM2551 was additionally supplemented with 0.6 μg/mL Insulin (SIGMA, Catalog number 19278), 1 μg/mL Hydrocortisone (SIGMA, Catalog number H0135), and 10 μM 1-Thioglycerol (SIGMA, Catalog number M6145).

TABLE 1
Cell line information

| | | | | | | | | Treat- |
|---|---|---|---|---|---|---|---|---|
| | Driver | | Source | | Medium | Medium | | ment |
| Cell line | mutations | Source | Cat Num | Medium | Vendor | Cat Num | #Cells | [h] |
| DLD-1 | KRAS, PIK3CA | ATCC | CCL-221 | RPMI | ThermoFisher | 22400-071 | 500 | 72 |
| HCT-116 | KRAS, PIK3CA | ATCC | CCL-247 | McCoy's 5A | ATCC | 30-2007 | 500 | 72 |
| LS-180 | KRAS, PIK3CA | ATCC | CCL-187 | EMEM | ATCC | 30-2003 | 800 | 72 |
| GP2d | KRAS, PIK3CA | ECACC | 95090714 | DMEM | ATCC | 30-2002 | 900 | 72 |
| SW480 | KRAS | ATCC | CCL-228 | RPMI | ATCC | 30-2001 | 700 | 72 |
| SW837 | KRAS | ATCC | CCL-235 | RPMI | ATCC | 30-2001 | 1250 | 72 |
| LoVo | KRAS | ATCC | CCL-229 | F-12K | ATCC | 30-2004 | 1250 | 96 |
| RKO | BRAF, PKI3CA | ATCC | CRL-2577 | EMEM | ATCC | 30-2003 | 500 | 72 |
| LIM2551 | BRAF, PKI3CA | CellBank | CBA-0170 | RPMI | ATCC | 30-2001 | 1000 | 72 |
| | | Australia | | | | | | |
| HT-29 | BRAF, PKI3CA | ATCC | HTB-38 | McCoy's 5A | ATCC | 30-2007 | 800 | 72 |
| OUMS-23 | BRAF | HSRRB | JCRB1022 | DMEM | ATCC | 30-2002 | 900 | 72 |
| LS411N | BRAF | ATCC | CRL-2159 | RPMI | ATCC | 30-2001 | 900 | 72 |
| COLO-205 | BRAF | ATCC | CCL-222 | RPMI | ATCC | 30-2001 | 800 | 72 |
| NCl-H508 | PKI3CA | ATCC | CCL-253 | RPMI | ATCC | 30-2001 | 1000 | 72 |
| COLO-320 | | DSMZ | ACC-144 | RPMI | ATCC | 30-2001 | 800 | 72 |

Cell lines were cultured in 37° C. and 5% CO₂ incubator and expanded in T-75 flasks. In all cases cells were thawed from frozen stocks, expanded through≥1 passage using 1:3 dilutions, counted and assessed for viability using a ViCell counter (Beckman-Coulter) prior to plating. To split and expand cell lines, cells were dislodged from flasks using 0.25% Trypsin-EDTA (GIBCO, Catalog number 25200). All cell lines were determined to be free of mycoplasma contamination as determined by a PCR detection methodology performed at Idexx Radil (Columbia, Mo., USA) and correctly identified by detection of a panel of SNPs.

Images were analyzed after adapting previously described methods (Horn, Sandmann et al. 2011) and using the Bioconductor package EBImage in R (Pau, Fuchs et al. 2010). Objects in both channels, DAPI (for Hoechst/DNA) and FITC (for Caspase 3/7), were segmented separately by adaptive thresholding and counted. A threshold for Caspase 3/7 positive objects was defined manually per cell line after comparing negative controls (DMSO) and positive controls (Staurosporine). By analyzing 17 additional object/nuclei features in the DNA channel (shape and intensity features) debris/fragmented nuclei were identified. To this end, per cell line the distributions of the additional features between positive controls (Staurosporine) and negative controls (DMSO) were compared manually. Features that could differentiate between the conditions (e.g., a shift in the distribution of a feature measurement comparing DMSO with Staurosporine) were used to define the ‘debris’ population versus the population of ‘viable’ nuclei. The debris counts were subtracted from raw nuclei counts. The resulting nuclei number was used as measure of cell proliferation (‘cell count’).

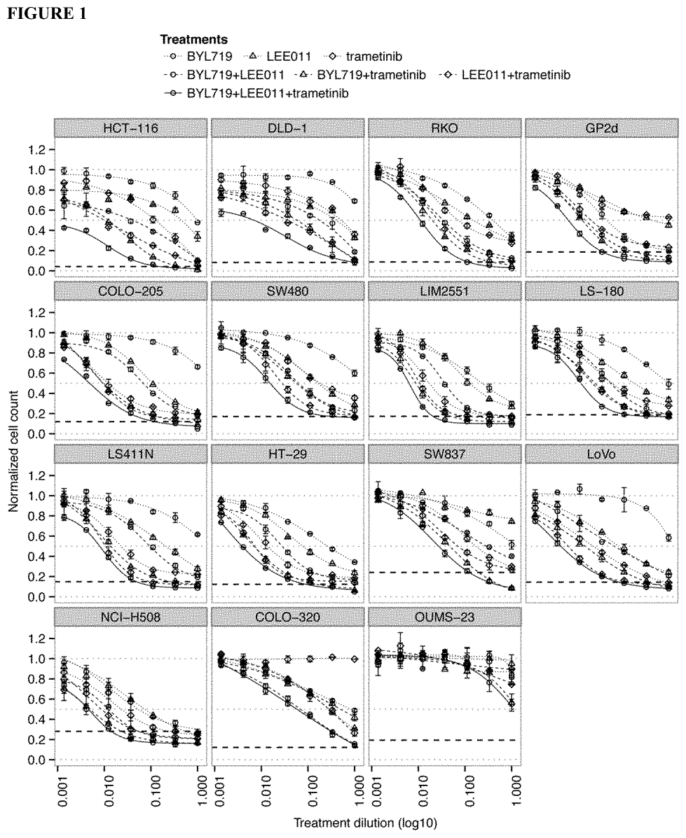

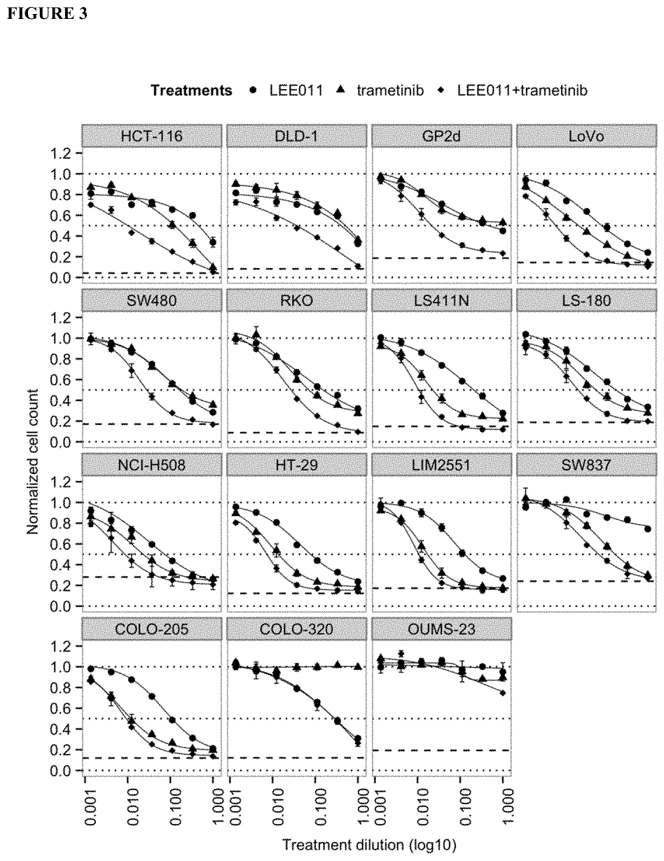

The compound's effect on cell proliferation was calculated from the cell counts of the treatments relative to the cell counts of the negative control (DMSO), in FIG. 1 and FIG. 3 denoted as ‘Normalized cell count’ (=‘xnorm’) on the y-axis. Synergistic combinations were identified using the highest single agent model (HSA) as null hypothesis (Berenbaum 1989). Excess over the HSA model predicts a functional connection between the inhibited targets (Lehar, Zimmermann et al. 2007, Lehar, Krueger et al. 2009). The model input were inhibition values per drug dose:

I=1−xnorm
I: inhibition
xnorm: normalized cell count (median of three replicates)

At every dose point of the combination treatment the difference between the inhibition of the combination and the inhibition of the stronger of the two single agents was calculated (=model residuals). To favor combination effects at high inhibition the residuals were weighted with the observed inhibition at the same dose point. The overall combination score C of a drug combination is the sum of the weighted residuals over all concentrations:

C=Σ_{Cone} (I_{data}*(I_{data}−I_{model}))
I_{data}: measured inhibition
   I_{model}: inhibition according to HSA null hypothesis

Robust combination z-scores (z_{C}) were calculated as the ratio of the treatments' combination scores C and the median absolute deviation (mad) of non-interacting combinations:

z_{C}=C/mad(C_{zero})
C_{zero}: combination scores of non-interacting combinations z_{C} is an indicator for the strength of the combination with:

z_{C}≥3: synergy

3>z_{C}≥2: weak synergy

z_{C}<2: no synergy

IC50 is the compound concentration that results in 50% of the cell counts relative to DMSO. IC50 calculations (see Table 2 and Table 3) were done using the DRC package in R (Ritz and Streibig 2005) and fitting a four-parameter log-logistic function to the data.

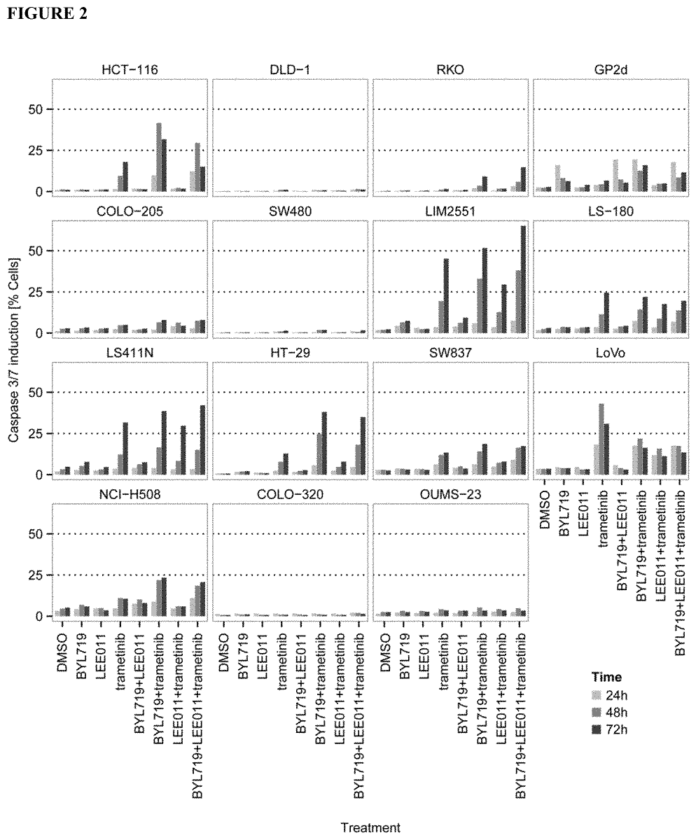

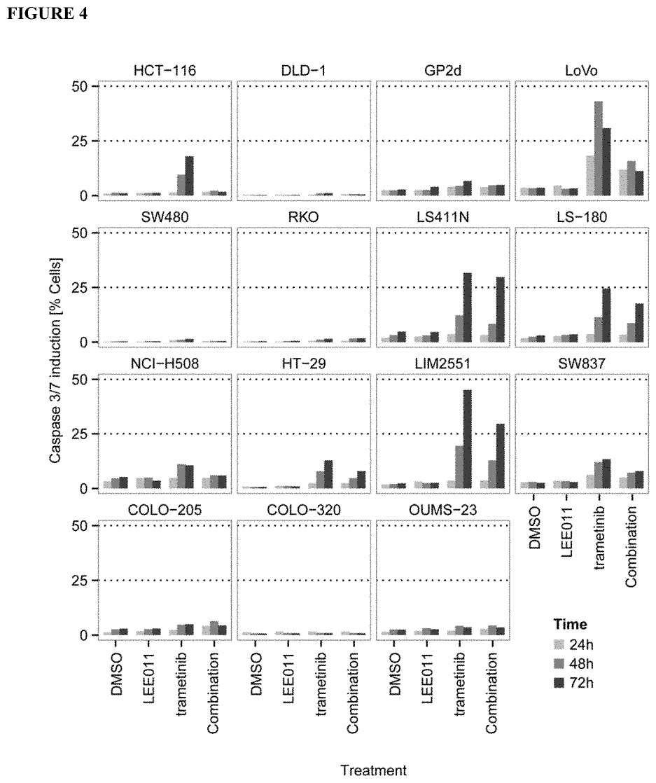

The compound's effect on apoptosis was determined by calculating the percentage of cells with activated Caspase 3/7 per treatment and time point relative to the raw cell counts (before subtraction of debris) (y-axis in FIG. 2 and FIG. 4). Cell counts at time points that were not experimentally measured were obtained by regression analysis by fitting a linear model for log-transformed cell counts at day 0 and the end of the treatment (assuming exponential cell growth).

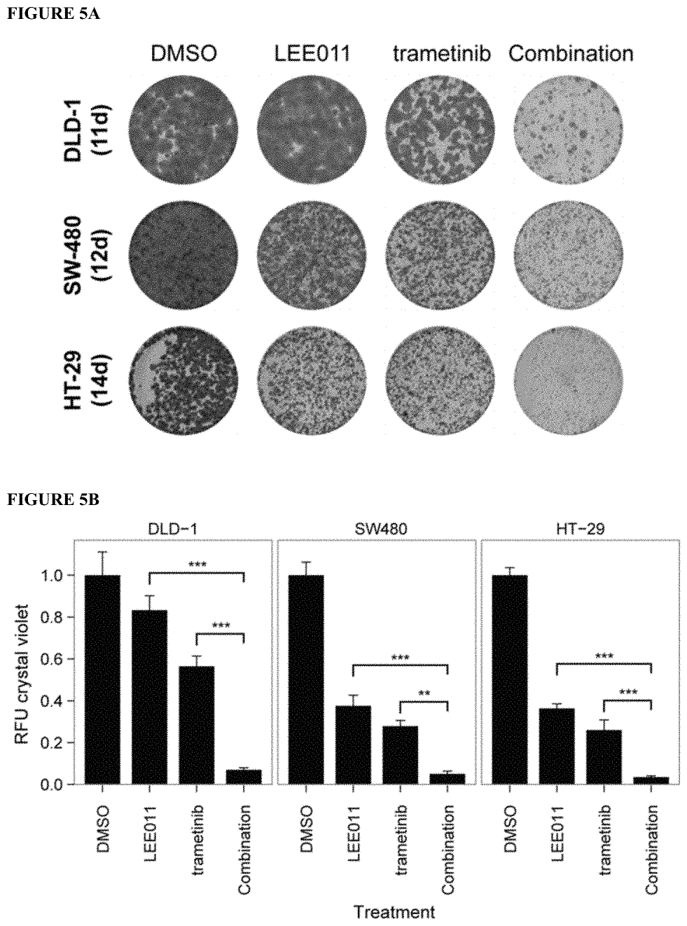

For colony formation assays (FIG. 5a) cells were plated in 1 mL medium in 12-well tissue culture-treated plates (Costar, Catalog number 3513): for DLD-1 1000 cells/well, for SW-480 1500 cells/well, and for HT-29 2,500 cells/well. Cells were grown for 24 h before addition of compounds, and treatments were refreshed every 72 h (in fresh medium) for up to 14 days using a HP D300 Digital Dispenser (Tecan). At the end of the treatment, cells were washed in PBS once, fixed and stained for 30 minutes at room temperature using a solution containing 4% PFA (Electron Microscopy Sciences, Catalog number 15714) and 2 mg/mL Crystal Violet (EMD, Catalog number 192-12), and washed 3 times with water. Plates were dried overnight and the scanned using an Odyssee imager (Licor). ImageStudio software (Licor) was used to quantify the crystal violet signal for FIG. 5a.

### Example 1: The In Vitro Effect on Proliferation of Combining the CDK4/6 Inhibitor LEE011 (also Known as “Ribociclib”) with the MEK Inhibitor Trametinib, and with the PIK3CA Inhibitor BYL719 (also Known as “Alpelisib”) in Colorectal Cancer Cell (CRC) Lines.

To test the effect of the combination of LEE011, trametinib and BYL719 on cell proliferation, cells were plated in black 384-well microplates with clear bottom (Matrix/Thermo Scientific, Catalog number 4332) in 50 μL media per well at cell densities between 500 and 1250 cells/well (Table 1) and allowed to incubate at 37 degrees, 5% CO₂ for 24 h. After 24 h one 384-well plate per cell line was prepared for cell counting by microscopy (see below) without receiving treatment (=‘baseline’). The other cell plates were treated by transferring 25 nL of the 2000× compound from drug master plates using an ATS acoustic liquid dispenser (ECD Biosystems) and resulting in a final 1× concentration. BYL719 was used over a final concentration range of 13 nM-10 μM, LEE011 was used over a final concentration range of 13 nM-10 μM, and trametinib was used over a final concentration range of 0.4 nM-0.3 μM (7 1:3 dilution steps). In order to assess the effect of the triple combination all individual compounds, all three pair wise combinations, and the triple combination were tested in the same experiment.

Pair wise combinations and the triple combination were tested at a fixed ratio of 1:1 (for drug pairs) and 1:1:1 (for the drug triple) at each dilution resulting in 7 combination conditions per treatment. Additionally, negative controls (DMSO=‘vehicle’) and positive controls (Staurosporine=killing cells, 7-point 1:2 dilution series for a dose range of 16 nM-1 μM) were transferred as treatment controls, and compounds with no efficacy in the cell lines tested were used in combinations with BYL719 and LEE011 as combination controls (combinations that do not exceed the efficacy of the more efficacious single agent=‘non-interacting’ combinations). After compound addition 50 nL of 2 mM CellEvent Caspase-3/7 Green Detection Reagent (ThermoFisher, Catalog number C10423) were added to one of the three replicates using the HP D300 Digital Dispenser (Tecan). Caspase 3/7 induction was measured as a proxy for apoptosis induced by the treatments. Cells were treated for 72 h to 96 h depending on their doubling time (Table 1), and Caspase 3/7 activation was measured every 24 h by microscopy using an InCell Analyzer 2000 (GE Healthcare) equipped with a 4X objective and FITC excitation/emission filters. At the end of the treatment, cells were prepared for cell counting by microscopy. Cells were fixed and permeabilised for 45 minutes in 4% PFA (Electron Microscopy Sciences, Catalog number 15714), 0.12% TX-100 (Electron Microscopy Sciences, Catalog number 22140) in PBS

(Boston Bioproducts, Catalog number BM-220). After washing cells three times with PBS their DNA was stained for 30 minutes with Hoechst 33342 (ThermoFisher, Catalog number H3570) at a final concentration of 4 μg/mL. Cells were washed three times with PBS and then plates were heat-sealed using a PlateLoc (Agilent Technologies) with aluminum seals (Agilent Technologies, Catalog number 06644-001) and stored at 4° C. until imaging. All cells per well/treatment were captured in a single image by fluorescence microscopy using an InCell Analyzer 2000 (GE Healthcare) equipped with a 4X objective and DAPI excitation/emission filters.

The efficacies of a PIK3CA inhibitor BYL719, a CDK4/6 inhibitor LEE011, and a MEK inhibitor trametinib were assessed individually and in combination in a total of 15 colorectal cancer cell lines. Cell lines were mutant in KRAS, BRAF, and/or PIK3CA, or wild type for all 3 genes (Table 1). BYL719 and LEE011 showed mostly micromolar IC50 values, with LEE011 being more potent across the cell lines tested. BYL719 only reached an IC50 in 7/15 cell lines, while LEE011 reached an IC50 in 13/15 cell lines. Trametinib had nanomolar to sub-micromolar IC50s in all but 3 cell lines (GP2d, COLO-320, and OUMS-23) (FIG. 1 and Table 2). The triple combination (LEE011+trametinib+BYL719) caused synergistic inhibition (according to the HSA model) over the drug pairs in 10/15 cell lines with different strengths, as well as weakly synergistic inhibition in 3/15 cell lines (Table 2). The triple combination does not more strongly induce apoptosis (assessed by measuring Caspase 3/7 induction) compared to the pair wise combinations (FIG. 2). Collectively, combined inhibition of PIK3CA, CDK4/6, and MEK in CRC may provide an effective therapeutic modality capable of improving responses compared to each of the single agents and may lead to more durable responses in the clinic.

TABLE 2
Single agent IC50 values for each compound and synergy z-score
measurements for the combination of LEE011, trametinib,
and BYL719.

| | IC50 | IC50 | IC50 | |
|---|---|---|---|---|
| Cell | BYL719 | LEE011 | Trametinib | Synergy z-score (z_{c}) |
| HCT-116 | 9.8 | 4.5 | 0.038 | 12.7 |
| DLD-1 | >10 | 4.1 | 0.153 | 10.2 |
| RKO | 3.9 | 1.5 | 0.021 | 6.7 |
| GP2d | 0.5 | 4.5 | >0.3 | 5.9 |
| COLO-205 | >10 | 1.1 | 0.004 | 5.1 |
| SW480 | >10 | 1.6 | 0.055 | 4.8 |
| LIM2551 | 2.3 | 1.3 | 0.005 | 4.7 |
| LS-180 | >10 | 1.8 | 0.018 | 4.6 |
| LS411N | >10 | 2.1 | 0.007 | 3.6 |
| HT-29 | 2.7 | 0.8 | 0.004 | 3.5 |
| SW837 | >10 | >10 | 0.051 | 2.7 |
| LoVo | >10 | 0.8 | 0.007 | 2.6 |
| NCI-H508 | 0.5 | 0.7 | 0.007 | 2.2 |
| COLO-320 | 8.3 | 2.8 | >0.3 | 1 |
| OUMS-23 | >10 | >10 | >0.3 | 0.5 |

### Example 2: The In Vitro Effect on Proliferation of Combining the CDK4/6 Inhibitor LEE011 with the MEK Inhibitor Trametinib in Colorectal Cancer Cell (CRC) Lines.

To test the effect of the combination of LEE011 and trametinib on cell proliferation, cells were plated in black 384-well microplates with clear bottom (Matrix/Thermo Scientific, Catalog number 4332) in 50 μL media per well at cell densities between 500 and 1250 cells/well (Table 1) and allowed to incubate at 37 degrees, 5% CO₂ for 24 h. After 24 h one 384-well plate per cell line was prepared for cell counting by microscopy (see below) without receiving treatment (=‘baseline’). The other cell plates were treated by transferring 25 nL of the 2000X compound from drug master plates using an ATS acoustic liquid dispenser (ECD Biosystems), resulting in a final 1× concentration. LEE011 was used over a final concentration range of 13 nM-10 μM, and trametinib was used over a final concentration range of 0.4 nM-0.3 μM (7 1:3 dilution steps). For the combination of LEE011 with trametinib, the single agents were combined at a fixed ratio of 1:1 at each dilution resulting in 7 combination treatments. Additionally, negative controls (DMSO=‘vehicle’) and positive controls (Staurosporine=killing cells, 7-point 1:2 dilution series for a dose range of 16 nM-1 μM) were transferred as treatment controls, and compounds with no efficacy in the cell lines tested were used in combination with LEE011 and trametinib as combination controls (combinations that do not exceed the efficacy of the more efficacious single agent=‘non-interacting’ combinations). After compound addition, 50 nl of 2 mM CellEvent Caspase-3/7 Green Detection Reagent (ThermoFisher, Catalog number C10423) was added to one of the three replicates using the HP D300 Digital Dispenser (Tecan). Caspase 3/7 induction was measured as a proxy for apoptosis induced by the treatments. Cells were treated for 72 h to 96 h depending on their doubling time (Table 1), and Caspase 3/7 activation was measured every 24 h by microscopy using an InCell Analyzer 2000 (GE Healthcare) equipped with a 4× objective and FITC excitation/emission filters. At the end of the treatment cells were prepared for cell counting by microscopy. Cells were fixed and permeabilised for 45 minutes in 4% PFA (Electron Microscopy Sciences, Catalog number 15714), 0.12% TX-100 (Electron Microscopy Sciences, Catalog number 22140) in PBS (Boston Bioproducts, Catalog number BM-220). After washing cells three times with PBS their DNA was stained for 30 minutes with Hoechst 33342 (ThermoFisher, Catalog number H3570) at a final concentration of 4 μg/mL. Cells were washed three times with PBS and then plates were heat-sealed using a PlateLoc (Agilent Technologies) with aluminum seals (Agilent Technologies, Catalog number 06644-001) and stored at 4° C. until imaging. All cells per well/treatment were captured in a single image by fluorescence microscopy using an InCell Analyzer 2000 (GE Healthcare) equipped with a 4× objective and DAPI excitation/emission filters.

The efficacies of the CDK4/6 inhibitor LEE011 and a MEK inhibitor trametinib were assessed individually and in combination in a total of 15 colorectal cancer cell lines. Cell lines were mutant in KRAS, BRAF, and/or PIK3CA, or wild type for all 3 genes (Table 1). LEE011 as single agent inhibited the growth of all but two cell lines (SW837 and OUMS-23) with sub-micromolar to micromolar IC50 values (FIG. 3 and Table 3). Trametinib as single agent strongly inhibited the growth of all but 3 cell lines (GP2d, COLO-320, and OUMS-23) with nanomolar to sub-micromolar IC50 values (FIG. 3 and Table 3). The combination treatment caused synergistic inhibition (according to the HSA model) in 13/15 cell lines tested with different strengths (Table 3). Cell lines mutant for KRAS and PIK3CA or mutant for KRAS benefitted the most from the combination (Table 3). The combination does not more strongly induce apoptosis (assessed by measuring Caspase 3/7 induction) compared to the single agents, which might be a result of the cell-cycle arrest induced after inhibition of CDK4/6 (FIG. 4). Long-term colony formation assays for two KRAS mutant lines (DLD-1 and SW480) and one BRAF mutant line (HT-29) showed that the combination provides significantly better efficacy compared to each of the single agents (FIG. 5a and FIG. 5b). Collectively, combined inhibition of CDK4/6 and MEK in CRC may provide an effective therapeutic modality capable of improving responses compared to each of the single agents and lead to more durable responses in the clinic.

TABLE 3
Single agent IC50 values for each compound and synergy z-score
measurements for the combination of LEE011 and trametinib.

| Cell | IC50 LEE011 | IC50 Trametinib | Synergy z-score (z_{c}) |
|---|---|---|---|
| HCT-116 | 4.5 | 0.038 | 15.9 |
| DLD-1 | 4.1 | 0.153 | 12 |
| GP2d | 4.5 | >0.3 | 11.7 |
| LoVo | 0.8 | 0.007 | 11 |
| SW480 | 1.6 | 0.055 | 10.8 |
| RKO | 1.5 | 0.021 | 9 |
| LS411N | 2.1 | 0.007 | 8.8 |
| LS-180 | 1.8 | 0.018 | 7.9 |
| NCI-H508 | 0.7 | 0.007 | 7.4 |
| HT-29 | 0.8 | 0.004 | 6.8 |
| LIM2551 | 1.3 | 0.005 | 4.4 |
| SW837 | >10 | 0.051 | 4.2 |
| COLO-205 | 1.1 | 0.004 | 4.1 |
| COLO-320 | 2.8 | >0.3 | 0.1 |
| OUMS-23 | >10 | >0.3 | −0.1 |

1. A pharmaceutical combination comprising:
(a) a first compound having the structure of formula (I): 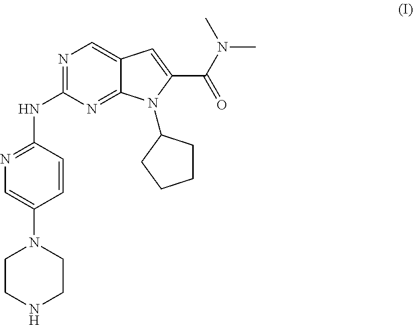 [SMILES: CN(C)C(=O)c1cc2cnc(Nc3ccc(N4CCNCC4)cn3)nc2n1C1CCCC1]
or a pharmaceutically acceptable salt or solvate thereof, and
(b) a second compound having the structure of formula (II): 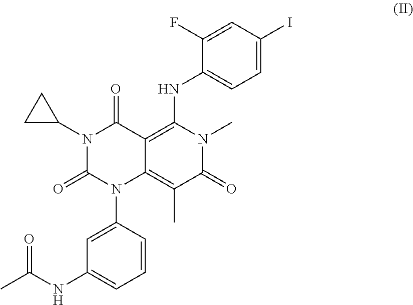 [SMILES: CC(=O)Nc1cccc(-n2c(=O)n(C3CC3)c(=O)c3c(Nc4ccc(I)cc4F)n(C)c(=O)c(C)c32)c1]
or a pharmaceutically acceptable salt or solvate thereof.

2. The pharmaceutical combination of claim 1, wherein the compound having the structure of formula (I), or a pharmaceutically acceptable salt or solvate thereof, and the compound having the structure of formula (II), or a pharmaceutically acceptable salt or solvate thereof, are in the same formulation.

3. The pharmaceutical combination of claim 1, wherein the compound having the structure of formula (I), or a pharmaceutically acceptable salt or solvate thereof, and the compound having the structure of formula (II), or a pharmaceutically acceptable salt or solvate thereof, are in separate formulations.

4. The pharmaceutical combination of claim 1, wherein the combination is for simultaneous or sequential administration.

5. The pharmaceutical combination of claim 1, further comprising a third compound having the structure of formula (III): 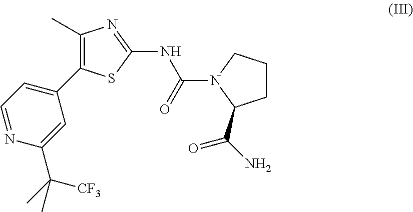 [SMILES: Cc1nc(NC(=O)N2CCC[C@H]2C(N)=O)sc1-c1ccnc(C(C)(C)C(F)(F)F)c1]
or a pharmaceutically acceptable salt or solvate thereof.

6. The pharmaceutical combination of claim 5, wherein the compound having the structure of formula (I), or a pharmaceutically acceptable salt or solvate thereof, the compound having the structure of formula (II), or a pharmaceutically acceptable salt or solvate thereof, and the compound having the structure of formula (III), or a pharmaceutically acceptable salt or solvate thereof, are in the same formulation.

7. The pharmaceutical combination of claim 5, wherein the compound having the structure of formula (I), or a pharmaceutically acceptable salt or solvate thereof, the compound having the structure of formula (II), or a pharmaceutically acceptable salt or solvate thereof, and the compound having the structure of formula (III), or a pharmaceutically acceptable salt or solvate thereof, are in 2 or 3 separate formulations.

8. The pharmaceutical combination of claim 5, wherein the combination is for simultaneous or sequential administration.

9. The pharmaceutical combination of claim 1, wherein the first compound is the succinate salt of the compound having the structure of formula (I).

10. A method for the treatment or prevention of cancer in a subject in need thereof, comprising administering to the subject a therapeutically effective amount of a pharmaceutical combination of claim 1.

11. The method of claim 10, wherein the cancer is selected from the group consisting of pancreatic cancer, breast cancer, mantle cell lymphoma, non-small cell lung cancer, melanoma, colorectal cancer, esophageal cancer, liposarcoma, multiple myeloma, T-cell leukemia, renal cell carcinoma, gastric cancer, glioblastoma, hepatocellular carcinoma, lung cancer, and rhabdoid tumor.

12. The method of claim 11, wherein the cancer is pancreatic cancer, breast cancer, or mantle cell lymphoma.

13. The method of claim 11, wherein the cancer is mantle cell lymphoma.

14. The method of claim 11, wherein the cancer is rhabdoid tumor.

15. The method of claim 11, wherein the cancer is colorectal cancer.

16. The method of claim 10, wherein the cancer is characterized by a PIK3CA mutation and/or a PIK3CA overexpression.

17. The pharmaceutical combination of claim 1, for use in the treatment or prevention of cancer.

18. The pharmaceutical combination of claim 1, for use in the manufacture of a medicament for the treatment or prevention of cancer.

19. The pharmaceutical combination of claim 17, wherein the cancer is selected from the group consisting of pancreatic cancer, breast cancer, mantle cell lymphoma, non-small cell lung cancer, melanoma, colorectal cancer, esophageal cancer, liposarcoma, multiple myeloma, T-cell leukemia, renal cell carcinoma, gastric cancer, glioblastoma, hepatocellular carcinoma, lung cancer, and rhabdoid tumor.

20. The pharmaceutical combination of claim 19, wherein the cancer is pancreatic cancer, breast cancer, or mantle cell lymphoma.

21. The pharmaceutical combination of claim 19, wherein the cancer is mantle cell lymphoma.

22. The pharmaceutical combination of claim 19, wherein the cancer is rhabdoid tumor.

23. The pharmaceutical combination of claim 19, wherein the cancer is colorectal cancer.

24. The pharmaceutical combination of claim 17, wherein the cancer is characterized by a PIK3CA mutation and/or PIK3CA overexpression.

25. Use of a pharmaceutical combination of claim 1 for the manufacture of a medicament for the treatment or prevention of cancer.

26. Use of a pharmaceutical combination of claim 1 for the treatment or prevention of cancer.

27. The use of claim 25, wherein the cancer is selected from the group consisting of pancreatic cancer, breast cancer, mantle cell lymphoma, non-small cell lung cancer, melanoma, colorectal cancer, esophageal cancer, liposarcoma, multiple myeloma, T-cell leukemia, renal cell carcinoma, gastric cancer, glioblastoma, hepatocellular carcinoma, lung cancer, and rhabdoid tumor.

28. The use of claim 27, wherein the cancer is pancreatic cancer, breast cancer, or mantle cell lymphoma.

29. The use of claim 27, wherein the cancer is mantle cell lymphoma.

30. The use of claim 27, wherein the cancer is rhabdoid tumor.

31. The use of claim 27, wherein the cancer is colorectal cancer.

32. The use of claim 27, wherein the cancer is characterized by a PIK3CA mutation and/or PIK3CA overexpression.

33. A pharmaceutical composition comprising:
(a) a first compound having the structure of formula (I): 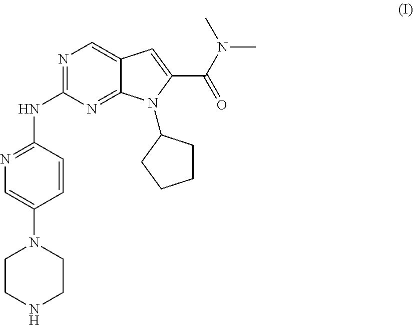 [SMILES: CN(C)C(=O)c1cc2cnc(Nc3ccc(N4CCNCC4)cn3)nc2n1C1CCCC1]
or a pharmaceutically acceptable salt or solvate thereof, and
(b) a second compound having the structure of formula (II): 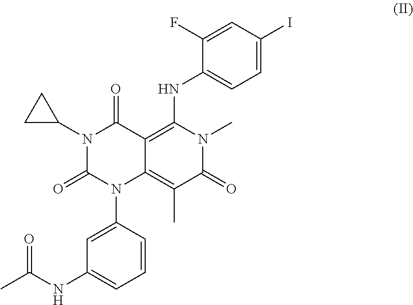 [SMILES: CC(=O)Nc1cccc(-n2c(=O)n(C3CC3)c(=O)c3c(Nc4ccc(I)cc4F)n(C)c(=O)c(C)c32)c1]
or a pharmaceutically acceptable salt or solvate thereof.

34. The pharmaceutical composition of claim 33, further comprising a third compound having the structure of formula (III): 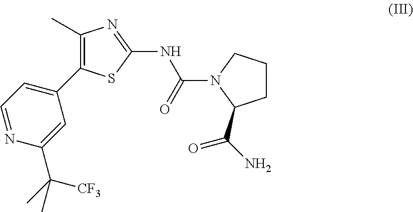 [SMILES: Cc1nc(NC(=O)N2CCC[C@H]2C(N)=O)sc1-c1ccnc(C(C)(C)C(F)(F)F)c1]
or a pharmaceutically acceptable salt or solvate thereof.

35. The pharmaceutical composition of claim 33, further comprising one or more excipients.
